(19) United States
(12) Patent Application Publication
(10) Pub. No.: US 2003/0176833 A1
(43) Pub. Date: Sep. 18, 2003

(54) System and method for fluid flow management

(76) Inventor: Ofer Libermann, Ramat Gan (IL)

Correspondence Address: LOWE HAUPTMAN GILMAN AND BERNER, LLP, 1700 DIAGONAL ROAD, SUITE 300 /310, ALEXANDRIA, VA, 22314, US

(21) Appl. No.: 10/333,606

(22) PCT Filed: Jul. 22, 2001
(86) PCT No.: PCT/IL01/00673

(30) Foreign Application Priority Data
Jul. 23, 2000 (IL) 137448

(51) Int. Cl. A61M 31/00

(52) U.S. Cl. 604/65

(57) ABSTRACT

A system and method for ensuring continuous fluid flow through a main conduit, the system including at least two fluid containers, each having a fluid outlet conduit couplable to the main conduit, an automatic closure device mounted on each fluid outlet conduit, and a controller for automatically sensing emptying of one of said fluid containers and automatically opening an automatic closure device on a full container, in response thereto.

### FIELD OF THE INVENTION

The present invention relates to systems and methods for fluid management in general and, in particular, to a system and method for accurately controlling fluid flow during and after a medical procedure.

### BACKGROUND OF THE INVENTION

During and after many surgical operations, such as urological endoscopic operations (such as transurethral resection of the prostate, or tumors TUR-P/T, Cytoscopy, P.C.N. (PerCutaneous Neparoscopy), and Ureteroscopy, and in various arthroscopic procedures, operative hysteroscopy, and liposuction, it is necessary to continuously flush the site with sterile fluids, in order to preserve the surgical area clear and maintain visibility throughout the operation. Generally, containers of a suitable irrigation fluid, such as Ringer solution, sterile water, normal saline, surbitolemanitol, 2% glycine, and so on, are used for this purpose. Conventional containers hold 3 liters each.

These containers are generally hung on suitable hangers above the operation table. Generally, they are connected in pairs to a single line of tubing, which brings the fluid to the site of the operation. The manifold leading to the containers from the main conduit leading to the site of the operation includes two tubes leading to each of the containers. A manual clamp is mounted on each tube, which permits opening and closing of the clamp by manual actuation of the clamp, which applies pressure to the tube. In this way, the tubing from each container can be opened and closed individually, generally one after the other.

The containers are generally formed of flexible plastic materials. Thus, it is possible, for those operations which require it, to apply external pressure to the containers, so as to increase the flow rate and pressure at the site of the operation. Alternatively, the main line can be connected to a peristaltic pump, in order to control the pressure and flow rate. In some of these operations, there is no need for pressure higher than 50 to 200 cm of water. In this case, it is customary to raise the containers and hang them close to the ceiling of the room, so as to create the desired pressure and flow by means of gravity.

In arthroscopic operations, the overall required fluid flow for a single operation can be as much as 12 liters and more. In urological operations and operative hysteroscopy, the overall fluid flow in a single operation can reach 40 liters and more.

Since a continuous fluid flow is required, the operating room team must be alert at all times in order to change fluid bags, as each one empties. In order to maintain continuity of flow, the team must exchange fluid bags precisely at the time the active container is empty. Generally, the two fluid containers or bags which are coupled to a single Y-manifold (or Y-SET) are not active at the same time, so as to avoid a long break when both of them are empty. Therefore, when the first container is empty, it must be closed, as by means of a clamp, and the clamp on the adjacent (full) container must be opened.

While the second container is active, and slowly being emptied, the first empty container is replaced by a fresh, full container. When the second container is empty, the clamp thereon is closed and the clamp on the new, full container is opened, as so on.

When the containers are mounted in a pressurized system, which is also hanging from the same hanger as the containers, each empty container must be disconnected from the pressurized system before removal. On the other hand, when the containers are raised above the patient, they must be lowered to a height at which a person can replace them, or an operating room team member must climb up on a ladder in order to reach the containers to switch them. When the main conduit is coupled to a peristaltic pump on its way to the patient, switching of the containers is the same as in other cases.

The various fluid supply systems described above, with all the conventional variations, does not permit satisfactory operation during this sort of operation. Since the response time of the operating room team (who are also responsible for performing other tasks during the operation) is generally after a container is empty, breaks in the fluid supply result. These breaks lengthen the time required for the operation, and, to some extent, endanger the life of the patient, since visibility of the surgeon is interrupted for a period of time.

During the operation, the fluids supplied as described above, which have collected particles of tissues, blood and fragments must be diligently removed from the patient. These fluids, which are removed from the patient's body, are generally collected in disposable containers holding about 3-4 liters, and thrown in the trash. These containers generally are coupled in series to a vacuum pump, and suction is created through them on the conduit from the containers to the site of the operation. When a container fills up, the suction action ceases, and the container must be switched with another. This occurs by transferring the end of the conduit exiting from the site of the operation from the full container to an adjacent empty container. As with fluid supply, fluid removal also creates pauses in the operation, which result from the human response time in the operating room environment.

For urological operations which take place in the bladder (such as, for instance, TUR-P/T), it is necessary to continuously flush the interior of the bladder, both during the operation and after, when the patient is no longer on the operating table. The object of this post-operative flushing is to flush out any blood from the incision, so that no adhesions or strictions will form in the urethra.

At present, this flushing is accomplished via a catheter inserted through the urethra. The catheter includes two fluid conduits—one provides a flow of sterile solution into the urethra, and the second removes the waste fluid from the bladder. Here, too, two or more fluid containers are hung from an infusion stand, and connected (in the same fashion as during an operation) to the inflowing conduit of the catheter. The waste removal conduit permits emptying of the bladder to a container, which can be located at the foot of the patient. A nurse must visit each patient and check the containers several times during the hours following an operation, to ensure that there is no blockage in the catheter and to switch empty solution containers with full containers.

The quantity and type of fluids used in post-operative flushing are similar to those used during an operation. Therefore, the nurse must check each patient many times in the hours following an operation. Since there are other patients, and the nursing staff cannot always arrive at the precise moment when one solution container empties completely, there often occurs a break or pause in the flushing fluid flow, and blood clots can form in the bladder. This causes blockage in the catheter, at which point, the nurse must unblock the catheter by injecting fluids near the blockage with a syringe. Thus, it will be appreciated, that many times, due to the work load of nurses, post-operative flushing is not satisfactory.

A further danger exists in TUR-P/T operations and operative hysteroscopy, known as “water intoxication”, or “TUR- syndrome”. This is a situation in which a portion of the flushing fluid is absorbed into the blood circulation, and upsets the electrolyte balance. In order to prevent this from occurring, the staff must determine, during the operation and during post-operative flushing, that the quantity of fluid entering the body cavity is identical to or smaller than the quantity leaving the body. The conventional method includes recording the number of containers (and partial containers) of solution flowing into the patient and the number of containers collected from the patient. This is accomplished, according to another method, by weighing the quantity of fluid entering the body and comparing it with the weight of the fluid leaving the body. Examples of these methods are described in U.S. Pat. No. 5,503,626 to Goldrath, and EP 956080 to FemrX among others. WO 97/16220 further includes means for indicating when a container has been changed, so as to permit zero-ing of the system.

In arthroscopic operations and operative hysteroscopy, in addition to containers hanging above the patient, which provide fluid flow into the patient by means of the force of gravity, and containers on which external pressure is exerted, so as to increase the flow, it is common to use a peristaltic pump to control the pressure and flow rate in a main conduit between the containers. See, for example, U.S. Pat. No. 6,010,454 to Chandler et al. Peristaltic pump manufacturers provide special tubing, which is suited to their individual pump. This tubing generally includes two conduits, which are coupled to two fluid containers. Clamps are placed on these conduits for opening and closing passage through the conduits. These two conduits converge into a single main conduit, which includes a flexible portion, which is suited to the operation of the peristaltic pump and a pressure monitor therein. This pressure monitor is coupled to the pump controller, which stops the action of the pump when the pressure in the conduit is higher than a pre-selected pressure.

Here, too, as in the case of similar pumps used in urology, breaks often occur, during the operation, when one container has emptied and it must be switched manually to the container standing next to it, for the same reasons as described above.

There is also known, as from U.S. Pat. No. 4,559,036 to Wunsch, apparatus for controlling intravenous solutions and medications. These include computerized means to provide precisely measured quantities of each of a plurality of fluids. The required quantities of the various solutions are programmed in advance for permitting passage of a pre-selected volume of fluid, only.

Accordingly, there is a long felt need for means for accurately and automatically controlling the switching of fluid bags, and it would be very desirable to have such a system which is operative both during surgical procedures and in post-operative care.

### SUMMARY OF THE INVENTION

The present invention provides a system and method for accurately and automatically controlling fluid flow into and out of a surgical site, and for ensuring continuous fluid flow through a conduit to a patient. In particular, the invention relates to automatic switching of fluid containers, or providing an early warning to a medical team that a last container is about to become empty, in order to prevent a break in the fluid flow.

It is possible to warm the fluids before delivery to the patient. It is also possible to provide a warning of absorption of a dangerous quantity of fluid during and after TUR-P operations and hysteroscopic operations. Furthermore, it is possible to use the system to monitor bleeding in the fluid flow or blockages in the catheter in a patient.

Thus, there is provided according to the present invention, a system for ensuring continuous fluid flow through a main conduit, the system including at least two fluid containers, each having a fluid outlet conduit couplable to the main conduit, an automatic closure device mounted on each fluid outlet conduit, and a controller for automatically sensing emptying of one of said fluid containers and automatically opening an automatic closure device on a full container, in response thereto.

According to one embodiment, the automatic closure device is an automatic clamping device. According to an alternative embodiment, the automatic closure device is an automatic selector valve.

There is also provided a method for ensuring continuous fluid flow through a main conduit, the method including coupling at least two fluid containers to the main conduit, each fluid container having a fluid outlet conduit, automatically sensing emptying of one of said fluid containers, and automatically opening an automatic closure device on a full container, in response thereto.

According to one embodiment, the automatic closure device is an automatic clamping device. According to an alternative embodiment, the automatic closure device is an automatic selector valve.

### BRIEF DESCRIPTION OF THE DRAWINGS

The present invention will be further understood and appreciated from the following detailed description taken in conjunction with the drawings in which:

FIG. 1 is a schematic illustration of a system for ensuring continuous fluid flow constructed and operative in accordance with one embodiment of the present invention;

FIG. 2 is a schematic illustration of a system for ensuring continuous fluid flow constructed and operative in accordance with an alternative embodiment of the present invention, including a variety of optional elements; and

FIG. 3 is a schematic illustration of a system for ensuring continuous fluid flow constructed and operative in accordance with a preferred embodiment of the present invention.

### DETAILED DESCRIPTION OF THE INVENTION

The present invention relates to a system and method for accurately and automatically controlling fluid flow into and out of a surgical site and, in particular, for the automatic switching of fluid containers, such as infusion bags, during and after various surgical procedures, so as to ensure a continuous fluid flow. The system is based on the use of sensors to determine whether various fluid containers are full or empty, and a computer controlled, automatic closure device on a tubing outlet from each fluid container, for automatically opening a full container when a previous container is empty. In the case of irrigation fluid, when the system detects that one container is empty, it automatically shuts the automatic closure device on the tube extending from that container and, simultaneously, opens the automatic closure device on another fluid container, thereby providing a continuous, steady flow of irrigation fluid.

Automatic closure devices, according to one embodiment of the invention, preferably include an automatic pinch valve, which presses forcefully on a conduit until it is completely sealed to fluid flow. They can include electromagnetic pinch valves, hydraulic pinch valves, pneumatic pinch valves, or any other automatic means of closing a plastic conduit.

According to an alternative preferred embodiment, the automatic closure device includes a selector valve, which automatically selects one of a plurality of inlets.

The invention permits a steady, continuous flow of fluids, even when it is necessary to switch or remove fluid containers. The system is useful during and after many surgical operations, such as urological endoscopic operations and in various arthroscopic procedures and operative hysteroscopy, which were described above.

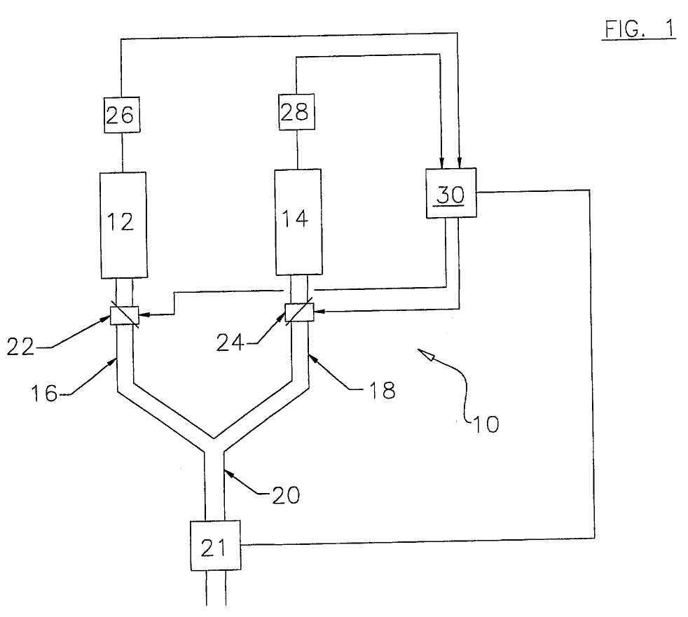

Referring now to FIG. 1, there is shown a schematic illustration of a system 10 for ensuring continuous fluid flow constructed and operative in accordance with one embodiment of the present invention. System 10 includes two fluid containers 12 and 14, holding a sterile fluid, such as for flushing a body cavity of a patient during an operation. Fluid containers 12 and 14 are coupled, via fluid outlet conduits 16 and 18, to a main conduit 20, which leads to the site of the operation. An automatic closure device, here shown as an automatic clamping device 22, 24 is coupled to each fluid outlet conduit 16, 18 so as to permit or prevent fluid flow therethrough. Automatic clamping devices 22 and 24 can be any type of pinch valve which is capable of sufficient providing pressure on the tubing of the fluid outlet conduits to prevent fluid flow therethrough, including, but not limited to electromagnetic, pneumatic, and hydraulic pinch valves.

At the beginning of the operation, fluid containers 12 and 14 are full. These containers are formed of a soft, flexible plastic material, and can include, for example, 3 liters of fluid. The containers are hung above the patient, as known. Each of containers 12 and 14 is coupled to a sensor 26, 28. Sensors 26 and 28 can be any suitable sensors for control of the quantity of liquid in the containers. Sensors 26 and 28 are arranged to determine by weight, volume, optical measurement, or in any other fashion, the quantity of fluid remaining in the associated container, or at least whether the respective container coupled to it is full or almost empty.

Sensors 26 and 28 are coupled to a controller 30. The sensor output corresponding to the state of the container is received by controller 30, which, in turn, is coupled to automatic clamping devices 22 and 24. Sensors 26 and 28 send signals periodically to the controller corresponding to the quantity of fluid in each container.

Alternatively, instead of separate sensors 26, 28, a single sensor or a flow meter 21 can be inserted in main conduit 20. Flow meter 21 is essentially a sensor, which sends signals to the controller corresponding to the quantity of fluid which has flowed out of the currently active container through the flow meter.

Operation of the system 10 of the invention is as follows. Two full containers 12, 14 are hung on the stand and coupled to main conduit 16. The automatic clamping device 22, mounted on outlet conduit 16 coupled to container 12, is opened by the controller 30 at the start of the flushing procedure. Automatic clamping device 24 mounted on outlet conduit 18 coupled to container 14 is closed. In this way, fluid from container 12, alone, flows into main conduit 20 and to the patient. Sensors 26 and 28 send signals to controller 30 corresponding to the quantity of fluid in each of containers 12 and 14. In the illustrated embodiment, the sensors are weight sensors, and send signals corresponding to the weight of each container. The controller determines whether the containers are full or close to empty.

When the controller 30 receives a signal from sensor 26 that container 12 is empty or almost empty, controller 30 sends a signal to automatic clamping device 24 mounted on outlet conduit 18 coupled to container 14, causing that automatic clamping device to open. Controller 30 also sends a signal to automatic clamping device 22 mounted on outlet conduit 16 coupled to container 12 to close. Then container 12 can be removed from the stand and replaced, if necessary.

It is a particular feature of the present invention that switching of the active container is carried out automatically by controller 30. Thus, there is no need for the operating room staff or the nursing staff to watch the flushing solution in order to change the active container. Furthermore, the switching is accomplished from outside the container, without any possibility of contaminating the sterile solution inside.

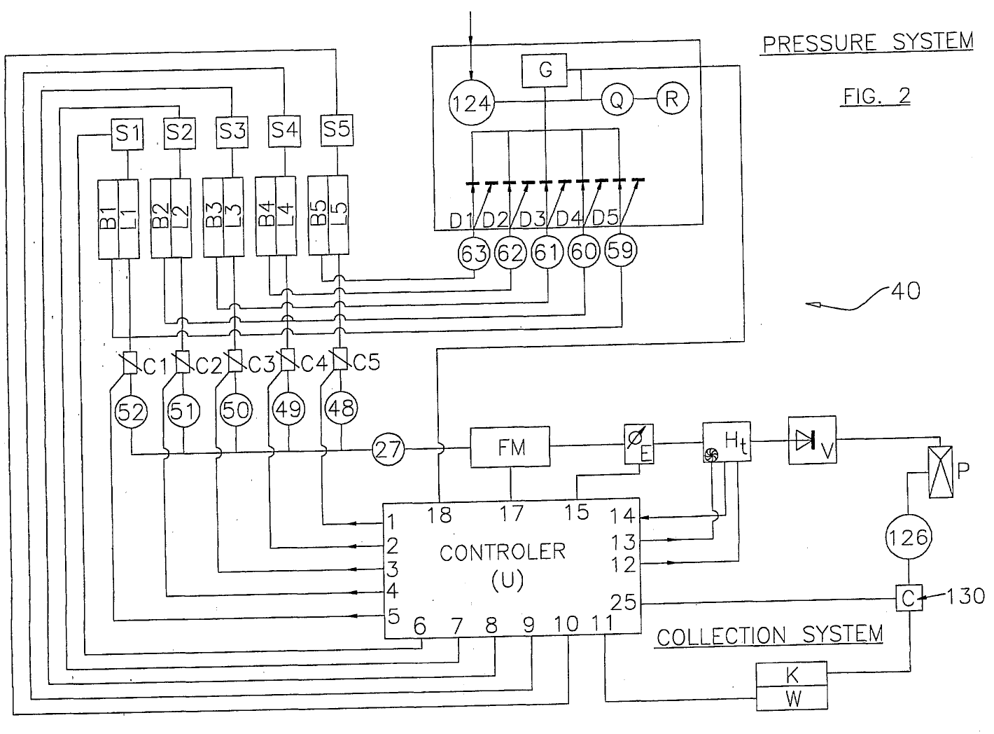

FIG. 2 is a schematic illustration of a system 40 for ensuring continuous fluid flow constructed and operative in accordance with an alternative embodiment of the present invention. System 40 includes all the possible elements of a system for ensuring fluid flow for urological and arthroscopic operations and post-operative care to a patient P. It will be appreciated that various elements illustrated in this system can be selected for inclusion or use, depending upon the particular needs of the specific care required by the patient.

System 40 includes a plurality of fluid containers L1, L2, L3, L4, L5, holding sterile fluids. Fluid containers L1 to L5 are coupled via fluid outlet conduits 48, 49, 50, 51, 52 to a main fluid conduit 27, which conducts the fluids to the patient P. Each of containers L1 to L5 may be provided with a pressure sleeve B1, B2, B3, B4, B5. Pressure sleeves B1 to B5 are compressed by air or gas and surround each of containers L1 to L5. They serve to increase the pressure inside the containers in order to increase the fluid supply and pressure at the site of the operation. Pressure sleeves B1 to B5 may be coupled to a source of pressure. Expanding and contracting is accomplished via gas conduits 59, 60, 61, 62, 63 and associated valves D1, D2, D3, D4, D5. The pressure source G can be an air compressor activated by the controller which selects the required pressure by means of a pressostat Q located next to a display of the controller to which a pressure meter R is attached. By means of pressure meter R, pressostat Q can be set. Alternatively, external pressure can be provided through a pipe 124 from an outside pressure source, instead of from pressure source G.

A sensor S1, S2, S3, S4, S5 is coupled to each fluid container and, via conductors U6, U7, U8, U9, U10, to a controller U. Controller U is programmed to respond when the quantity of fluid in the active container reaches about 50 cc. For ease of programming, all the values and physical parameters provided to controller U by the various sensors, such as weight in grams, or cc per unit time, can be converted, as required, relative to the specific weight of water.

Containers L1 to L5 are coupled by fluid outlet conduits 48, 49, 50, 51, 52 to a manifold, which becomes the main conduit 27 conducting the fluids to the patient. If desired, before reaching the patient, the fluids can be conducted through a heat exchanger H, for heating the flushing fluids, one-way valve V, mounted on main conduit 27 as close as possible to the patient, for preventing return flow of fluids from the patient to the system, and/or a flow limiter E for controlling the flow rate. Heat exchanger H can be any kind of heat exchanger which is safe for medical use. A heat exchanger is generally used in urological procedures, and serves to warm the fluids as they flow to the patient. Heat exchanger H is preferably controlled by controller 30, which includes a conductor U13 for activating a water circulation pump, a conductor U12 leading to a temperature sensor, and a conductor U14 for activating a heating element controlled by the temperature sensor.

Automatic clamping devices C1, C2, C3, C4, C5 are mounted on fluid outlet conduits 48 to 52. Preferably, automatic clamping devices C1 to C5 are normally closed, and serve to pinch the fluid outlet conduits so as to prevent fluid flow therethrough.

When the system is activated, controller U sends a signal via an electrical conductor U5 to open automatic clamping device C1, coupled to the first full container L1. This permits fluid from container L1 to flow through fluid outlet conduit 62 to main conduit 27 and, from there, to the patient.

When the signals received from sensor S1 at controller U via an electrical conductor U6 indicate that the quantity of fluid remaining in container L1 is less than about 50 cc, controller U causes automatic clamping device C1 to close, thereby cutting off fluid flow from container L1, and sends a signal via an electrical conductor U4 to open automatic clamping device C2, coupled to the second full container L2. This permits fluid from container L2 to flow through fluid outlet conduit 51 to main conduit 27 and, from there, to the patient.

In the same way, when the signals received from sensor S2 at controller U via an electrical conductor U7 indicate that the quantity of fluid remaining in container L2 is less than about 50 cc, controller U causes automatic clamping device C2 to close, thereby cutting off fluid flow from container L2, and sends a signal via an electrical conductor U3 to open automatic clamping device C3, coupled to the third full container L3, and so on. In this way, a continuous flow of fluid from one of the non-empty containers L1 to L5 flows at all times to main conduit 27 and, from there, to the patient.

The signals from sensors S1 to S5, which are received by controller U, are used to plan the next switching action, which can include consideration of the fact that some empty containers have been replaced with full containers and are now available for connection to the main conduit, when required. When only a single full container is left as the active container, controller U can activate an alarm system. For example, controller U can provide an audio-visual signal, when the fluid in the active container is reduced to about one half its volume. This signal can be silenced, by pressing on the controller display. Empty containers can be replaced with full ones, as required. Replacement bags can be hung on any available sensor S1 to S5. The controller U moves in a pre-determined order from container to container in order to switch to a full bag and maintain a continuous fluid flow through the main conduit. Preferably, this order is always from the lowest number available, to the highest. It will be appreciated that, after the last container has been emptied, controller U will open the first container, again, if it has been replaced with a full container.

On the screen of the controller, the number of the active container appears, together with the quantity of fluid remaining in that container at any given time. In addition, preferably, the total quantity of fluid which has passed through the system can be displayed. This information is stored in the controller's memory. The display may also contain, in addition to the conventional displays of “Start”, “Stop” and “Weighing”, a button permitting one to “Skip” directly to the next container.

The system 40 as described so far operates substantially in the same way as the embodiment of FIG. 1. Alternatively, it is possible to operate the system using a flow meter FM, which is located on or inside main conduit 27. Flow meter FM can be any suitable meter for measuring fluid flow, such as a turbine, photo electric flow meter, Doppler flow meter, mass flow meter, peristaltic flow meter, heat transition flow meter, or magnetic flow meter, among others. Flow meter FM is essentially a sensor, which sends signals to controller U corresponding to the quantity of fluid which has flowed out of the currently active container. These signals are received by controller U, via an electrical conductor U17. When they indicate passage of about 50 cc less than the total contents of the active container, controller U will send a signal to close the open automatic clamping device, and open the automatic clamping device on the next full container in line.

According to one embodiment of the invention, when the system is activated by a passive flow meter, as described above, sensors S1 to S5 can be simple sensors which only respond when an active container is totally empty. All the remaining functions are operative as described above.

In arthroscopic procedures, peristaltic pumps are generally employed in order to provide fluid to the joint, under controlled pressure and fluid flow. A precise, pre-defined quantity of fluid flows through a peristaltic pump during each rotation. A sensor, which is located close to the main axis of the pump, counts the number of rotations (preferably with a precision of 1/1000 rotation, or less) and sends a signal to the FM input of controller U, which will be programmed to multiply the known quantity of fluid which is pumped through the pump on each rotation by the number of actual rotations. When this quantity reaches a total equal to the entire contents of a container minus about 50 cc, or other selected quantity, the controller will be activated to switch containers, as described above regarding a flow meter FM. Such a peristaltic pump can be considered to be an active flow meter. (It will be appreciated that no heating or flow limiter is used in arthroscopic procedures.)

Another important aspect, particularly of TUR-P/T and hysteroscopy operations, is the balance of fluids. In these operations, it is important to measure the quantity of fluids flowing into the patient, and compare that quantity with the quantity of fluids flowing out of the patient, throughout the surgical procedure. The outflowing quantity must always be larger than the quantity of fluids supplied by the system into the patient, so as to prevent the development of TUR-Syndrome or water intoxication. In this case, the fluids from the patient, removed from the surgical site via a catheter through a conduit 126, are collected in a collection container K, which is coupled to a scale W or other sensor for detecting volume of fluid collected, which, in turn, is coupled to controller U. As stated above, controller U stores the total quantity of fluids supplied to the patient at any time. Thus, the quantity of fluid collected can easily be compared to the quantity supplied. In addition to its other characteristics, controller U is preferably arranged to display, on its display screen, the result of the balance of fluids, so that this data can be observed quickly and easily by the operating room staff. Preferably, when the difference reaches a pre-selected threshold, an alarm will sound or light will flash to indicate that the situation requires intervention by a staff member.

Post-operative flushing after TUR-P/T operations is generally carried out using a Folley Double Lumen type of catheter, which includes two conduits. Flushing fluids are provided through one of the conduits, in a manner similar to that during the operation (through conduit 27), while waste fluids are removed from the body cavity via the second conduit (through conduit 126). This form of flushing is known as continuous flow irrigation, and is carried out with automatic switching between fluid containers as described above with reference to FIG. 1. Furthermore, continuous flow irrigation also requires a proper balance of fluids, as described above.

According to one embodiment of the invention, an opacity meter 130, or other similar device, is provided to measure the clarity of the fluids removed from the patient's body, so as to determine whether there is still bleeding. When there is a lot of bleeding, it is necessary to provide irrigation fluid at a high rate. However, as the bleeding decreases, and the outflowing fluids become more clear and transparent, less irrigation fluid is required. Thus, controller U can receive signals from meter 130 via electrical conductor U25 indicating that a higher or lower flow rate is required, and controller U will activate a flow limiter E (via electrical conductor U15) to adjust the rate of flow of irrigation or flushing fluids to the patient. Flow limiter E can be any suitable device for selectively and controllably changing the fluid flow through main conduit 27, such as by applying pressure to the conduit. Preferably, the controller U is arranged to activate an alarm in the event that the quantity of fluid reaching the collection container K does not increase or decrease in accordance with the change in quantity of irrigation fluid supplied to the patient.

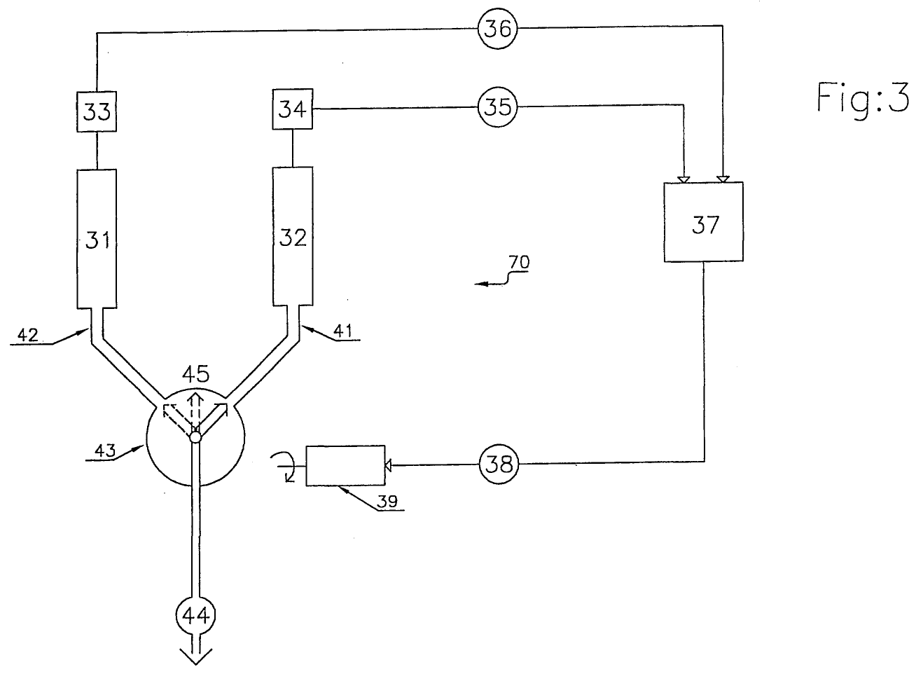

Referring now to FIG. 3, there is shown a schematic illustration of a system 70 for ensuring continuous fluid flow constructed and operative in accordance with another embodiment of the present invention. System 70 is substantially similar to system 10, but the automatic closure device is a single selector valve coupled to all the fluid containers, instead of a separate clamping device coupled to each fluid container.

System 70 includes a plurality of fluid containers, here shown as two fluid containers 31 and 32, each holding a sterile fluid, such as for flushing a body cavity of a patient during an operation. Fluid containers 31 and 32 are coupled via fluid outlet conduits 41 and 42 to a main conduit 44, which leads to the site of the operation. An automatic closure device, here shown as an automatic selector valve 43 is coupled between each fluid outlet conduit 41, 42 and the main conduit 44, so as to permit or prevent fluid flow from a selected fluid outlet conduit to the main conduit. Automatic selector valve 43 can be any type of selector valve which is capable of securely closing the tubing of the fluid outlet conduits to prevent fluid flow therethrough, and is preferably a mechanical selector valve. Selector valve 43 can be formed of biocompatible plastic, which is reusable after re-sterilization in a steam autoclave, or preferably formed of biocompatible single use plastic.

At the beginning of the operation, fluid containers 31 and 32 are full. Each of containers 31 and 32 is coupled to a respective sensor 33, 34, as described with regard to FIG. 1. Sensors 33 and 34 are arranged to determine by weight, volume, optical measurement, or in any other fashion, the quantity of fluid remaining in the associated container, or at least whether the respective container coupled to it is full or almost empty, and are preferably load cells, or other weight measuring device. According to an alternative embodiment, all the fluid containers are coupled to a single sensor unit, which determines, such as from relative position, the amount of fluid remaining in each container.

Sensors 33 and 34 are coupled to a controller 37, as by cables 35 and 36. The sensor output corresponding to the state of the container is received by controller 37 which, in turn, is drivingly coupled, as by a cable 38, to actuation element 39, such as a motor, for actuation of selector valve 43. Sensors 33 and 34 send signals periodically to the controller corresponding to the quantity of fluid in each container.

Operation of system 70 is substantially the same as that of system 10. Two (or more) full containers 31, 32 are hung on the stand and coupled, via outlet tubing 42 and 41 and automatic selector valve 43 to main conduit. The automatic selector valve 43 is coupled, via an actuation element 39, to controller 37. At the start of operation, automatic selector valve 43 is in a stand-by position 45, wherein none of the containers is open for fluid flow to the main conduit. Controller 37 causes actuation element 39 to cause selector valve 43 to select a first fluid container, such as 31 (i.e., open the passageway between outlet tubing 42 and main conduit 44), at the start of the flushing procedure. When actuation element 39 is a motor, the motor shaft causes mechanical rotation of the selector valve from one container to another. It will be appreciated that, in this way, all the other fluid containers are closed, so fluid cannot flow therefrom, and fluid from container 31, alone, flows into main conduit 44 and to the patient. Sensors 33 and 34 send signals to controller 37 corresponding to the quantity of fluid in each of containers 31 and 32, and the controller determines whether the containers are full or close to empty. If desired, a button can be provided in controller 37 for manual return of the selector valve to the stand-by position.

When the controller 37 receives a signal from sensor 33 indicating that container 31 is empty or almost empty, controller 37 sends a signal which activates motor 39 to cause automatic selector valve 43 to select a next, full, fluid container, i.e., 32, and close the previous, now substantially empty, fluid container. Then container 31 can be removed from the stand and replaced, if necessary.

It will be appreciated that the invention is not limited to what has been described hereinabove merely by way of example. Rather, the invention is limited solely by the claims which follow.

1. A system for ensuring continuous fluid flow through a main conduit, the system comprising:
at least two fluid containers, each having a fluid outlet conduit couplable to the main conduit;
an automatic closure device coupled to each fluid outlet conduit;
a sensor, coupled to each fluid container, arranged to provide a signal corresponding to a quantity of fluid in each said fluid container; and
a controller coupled to said sensor for receiving a signal corresponding to emptying of one of said fluid containers, automatically closing an automatic closure device coupled to said emptying container, and automatically opening an automatic closure device coupled to a full container, in response thereto.

2. The system according to claim 1, comprising a plurality of fluid containers and a plurality of sensors, one sensor being coupled to each fluid container, said controller being coupled to each of said sensors for receiving signals corresponding to a quantity of fluid in said fluid containers.

3. The system according to claim 1 or 2, comprising a plurality of fluid containers and a single sensor coupled to all of said fluid containers, said controller being coupled to said sensor for receiving signals corresponding to a quantity of fluid in said fluid containers.

4. The system according to any of the preceding claims, wherein said automatic closure device includes an automatic clamping device mounted on each fluid container and coupled between said fluid container and said main conduit, and coupled to said controller.

5. The system according to any of claims 1 to 3, wherein said automatic closure device includes an automatic selector valve coupled between each fluid container and said main conduit, and coupled to said controller.

6. The system according to claim 5, wherein said automatic selector valve is a mechanical valve, and said controller is coupled to a motor which is drivingly coupled to said mechanical valve.

7. The system according to any of the preceding claims, and further comprising at least two sleeves, one mounted around each fluid container for providing increased pressure to the contents of said fluid container.

8. The system according to claim 4, wherein said sensor includes a flow meter mounted in said main conduit for measuring fluid flow therethrough.

9. The system according to claim 4 or 5, wherein said sensor includes a peristaltic pump for measuring fluid flow therethrough.

10. The system according to any of claims 1 to 5, wherein said sensor includes a load meter for measuring weight of said container.

11. The system according to any of the preceding claims, further comprising a heat exchanger for heating fluid flowing through the main conduit.

12. The system according to any of the preceding claims, further comprising a flow limiter coupled to said controller for limiting the flow of fluid through the main conduit.

13. The system according to any of the preceding claims, further comprising a one-way valve for preventing return of fluid from a patient into the system.

14. The system according to any of the preceding claims, further comprising an alarm arranged to provide a warning of absorption of a dangerous quantity of fluid during and after operations.

15. The system according to any of the preceding claims, further comprising:
means for delivering fluid through the main conduit to a site;
means for removing fluid from said site;
means for measuring fluid removed from said site; and
means for comparing fluid delivered to said site with fluid removed from said site.

16. A method for ensuring continuous fluid flow through a main conduit, the method including:
coupling at least two fluid containers to the main conduit, each fluid container having a fluid outlet conduit;
automatically sensing emptying of one of said fluid containers by a sensor coupled to said container;
automatically closing an automatic closure device coupled to said emptying container in response thereto; and
automatically opening an automatic closure device coupled to a full container in response thereto.

17. The method according to claim 16, wherein said automatic closure device includes an automatic clamping device mounted on each fluid container and coupled between said fluid container and said main conduit, and coupled to said controller.

18. The method according to claim 16, wherein said automatic closure device includes an automatic selector valve coupled between each fluid container and said main conduit, and said controller is drivingly coupled to said automatic selector valve.

19. The method according to claim 18, wherein said steps of automatically closing and automatically opening comprise causing said controller to actuate a motor to activate said automatic selector valve.

20. The method according to claim 16, further comprising the step of warming fluid in the main conduit before delivery.

21. The method according to any of claims 16-20, further comprising the step of providing a warning of absorption of a dangerous quantity of fluid during fluid flow supply.

22. The method according to any of claims 16 to 21, further comprising the steps of:
delivering fluid through the fluid conduit to a site;
removing fluid from said site;
measuring said fluid removed from said site; and
comparing said fluid delivered to said site with said fluid removed from said site.

23. The method according to claim 17, wherein the step of automatically sensing includes sensing fluid flow through a flow meter.

24. The method according to claim 17 or 18, wherein the step of automatically sensing includes sensing fluid flow through a peristaltic pump.

25. The method according to any of claims 16 to 22, wherein the step of automatically sensing includes sensing weight of said containers.
